(19) United States
(12) Patent Application Publication
(10) Pub. No.: US 2007/0163703 A1
(43) Pub. Date: Jul. 19, 2007

(54) Card and mailing incorporating the card and system and method for producing the same

(76) Inventor: John M. Romita, Crystal Lake, IL (US)

Correspondence Address: Patents+TMS;A Professional Corporation, 2849 W. Armitage Ave., Chicago, IL, 60647, US

(21) Appl. No.: 11/653,577

(22) Filed: Jan. 16, 2007

Related U.S. Application Data
(60) Provisional application No. 60/760,238, filed on Jan. 19, 2006.

(51) Int. Cl. A61F 13/15

(52) U.S. Cl. 156/227

(57) ABSTRACT

A card and a mailing assembly incorporating the card and a system and a method for producing the same are provided. The system and the method produces the mailing assembly incorporating the card in a single manufacturing process. The system and the method have a card formed from a single web of a first material. The card is connected to, secured to and/or removably attached to a carrier which is formed from the single web. The card is secured to, connected to and/or removably attached to a carrier to form the mailing assembly. An identifier is incorporated between the layers of the card and/or on a top side and/or a bottom side of the card. A number is embossed on the card. The card and/or the carrier has a second material on and/or connected to the card and/or the carrier. A three-ply card is formed if the single web of material is folded over the second material. If the second material is cold foil having two layers, one layer of the foil may be transferred to the card.

This application claims the benefit of U.S. Provisional Application Ser. No. 60/760,238, filed Jan. 19, 2006.

### BACKGROUND OF THE INVENTION

The present invention generally relates to a card and a mailing incorporating the card and a system and a method for producing the same. More specifically, the present invention relates to a system and a method for producing a single-ply or multi-ply card from a single web of material. The card may be a promotional card, an identification card, a credit card, a membership card, a game piece and/or the like. The card may be incorporated into, connected to, secured to and/or removably attached to a carrier to form a mailing assembly.

The card and/or the carrier may be formed from the same single web of material. The card may be a membership card, a credit card, a discount card, a business card, a trading card, an authorization card, a coupon, a preferred customer card, an identification card, a novelty card, a ticket, a game piece and/or the like. The card may be incorporated into and/or formed into a mailing, such as, for example, a direct mailing, a magazine, a newspaper or the like.

Further, the card may have a layer, for example, a laminate such as, for example, a polypropylene or polyester film. As a result, the card may be durable and/or tear-resistant. Still further, the card may have an identifier, such as, for example, a radio frequency identification tag, a smart chip, a magnetic strip, a bar code and/or the like.

The card and/or the carrier may have an adhesive, such as, for example, a hot melt adhesive, an ultraviolet pressure sensitive adhesive and/or an ultraviolet curable adhesive. The card and/or the carrier may have personal imaging, such as, for example, a name and/or an address of a recipient. Imaging of the card and the carrier and/or steps for producing the card and/or the carrier may occur in a single-pass manufacturing process.

It is, of course, generally known for advertisers and marketing firms to use printing and finishing as a medium to promote products and/or services to the general public. For example, it is generally known that a credit card company may attach a personalized credit card to a letter. The credit card is generally formed of plastic and/or prepared from a manufacturing process different from the letter. The credit card may be attached to the letter which is mailed to the recipient. The letter is then folded and inserted into an envelope to be mailed. However, credit cards made of plastic are costly to produce and/or to mail. Furthermore, the plastic credit card may be produced by a different and/or a separate process from the letter. As a result, the credit card must be matched to the letter corresponding to the credit card. Accordingly, the credit card attached to the letter may require multiple manufacturing passes. Multiple manufacturing passes are costly, time consuming and frequently result in the credit card being mismatched with the letter.

Further, it is also generally known for commercial printers to produce products distributed by mail and/or inserted into newspapers, magazines or other periodicals. Commercial printers produce promotional cards which are personalized to a recipient. In use, the promotional card may be attached to a carrier to form a mailing assembly. Generally, each component of the mailing assembly is printed and/or is delivered from the manufacturing press as a flat sheet. The flat sheet may be directed to a finishing line remote to the manufacturing line where cutting, folding, gluing and/or personalization may occur. When the mailing assembly has multiple components, such as a promotional card attached to the carrier, generally the carrier and the promotional card may be produced at different times and/or different locations. The card and the carrier may be personalized separately at different times and/or locations. Then, the card and the carrier are matched during another process at a finishing line. The carrier may be cut and/or folded again to form the mailing assembly. As a result, the mailing assembly may have components produced from one or more processes at various times. Producing the mailing assembly by one or more processes is costly, time consuming and results in mismatching of the card and the carrier.

Further, it is generally known to produce a single-ply tag or a label attachable to a letter in a single manufacturing pass. The tag or label and corresponding letter are printed and/or imaged. The tag is die-cut from a web of material but may not be removed from the material. The tag is spot glued with a hot melt adhesive onto the letter before the tag is removed from the web. The web surrounding the tag is removed via a trim removal system. A single-ply label or a tag is often flimsy and easily torn. Specifically, in the Autumn of 2003, Ron Hancock, a former employee of Communisis, disclosed, in Broadview, Ill., a single-ply advertising packet having a single-ply card adhered to a single-ply ribbon. The card and the ribbon were produced from an in-line process. The card was die-cut from the ribbon and left within the ribbon (hereinafter “matrix”). The card was spot glued with the hot melt adhesive onto the ribbon before the matrix was removed.

Still further, it is generally known to produce a promotional card and a carrier from a single sheet of paper. The promotional card and the carrier are printed on the top side and the bottom side simultaneously. After the sheet is printed, the sheet moves to a finishing line. The sheet may be cut into a promotional card and a carrier. An ultraviolet coating is applied over the promotional card and/or the carrier for gloss and for protection. The ultraviolet coating is applied before the promotional card and/or the carrier are folded. The promotional card and/or the carrier are folded so that the ultraviolet coating is contained on the top and the bottom of the promotional card. The carrier is directed to a gluer to apply the hot glue spot adhesive at a spot on the carrier. The card is attached to the carrier at the spot via the adhesive. The promotional card is die cut, but is not removed from the remaining sheet (hereinafter “the matrix”). The matrix is removed and directed to a paper shredder. The carrier is cut to a final size and folded for mailing. However, the promotional card may be easily torn. Additionally, the promotional card may be damaged during the mailing process by, for example, rainwater. If the promotional card is damaged, the promotional card may be incapable of use after delivery to a recipient.

A need, therefore, exists for a system and a method for producing a card removably attached to a carrier from a single web of material. Additionally, a need exists for a card produced from a web of material which is tear-resistant. Further, a need exists for a system and a method for producing a card having a laminate in a single manufacturing process. Still further, a need exists for a system and a method for producing a card having an identifier in a single manufacturing process. Moreover, a need exists for a card made from a web of material and laminated to emulate a plastic card. In addition, a need exists for a system and a method for producing a laminated card and carrier which can form a mailing. Furthermore, a need exists for a card that provides protection from damaging by liquids, such as, for example, water, coffee or the like. In addition, a need exists for a system and a method for producing a mailing having a laminated multi-ply card from a single web of material. Further, a need exists for a system and a method for producing a promotional mailing having a card which is laminated and removably attached to a carrier. Still further, a need exists for a system and a method for producing a promotional mailing having a card with a laminate adhered to the card by a hot melt adhesive. Still further, a need exists for a system and a method for producing a promotional mailing having a card with a laminate adhered to the card by an ultraviolet pressure sensitive adhesive. Still further, a need exists for a system and a method for producing a promotional mailing having a card with a laminate adhered to the card by an ultraviolet curable adhesive.

### SUMMARY OF THE INVENTION

The present invention provides a card and a mailing incorporating the card and a system and a method for producing the same. More specifically, the present invention provides a system and a method for producing a card and/or a carrier from a single web of a first material. The first material may be, for example, paper, cardboard, cray paper and/or the like. One or more cards and/or one or more carriers may be formed from the single web. The cards and/or the carriers may form one or more mailing assemblies. The cards may be, for example, promotional cards, membership cards, game pieces, credit cards and/or the like. The carrier may be folded to create a mailing assembly having one or more pages. The card may be attached to the carrier via an adhesive. The mailing assembly may be, for example, a direct mailing, a magazine, a newspaper and/or the like.

The card and/or the carrier may be printed, imaged and/or screened on a top side and a bottom side simultaneously. The card and/or the carrier may be personalized to a recipient of the mailing assembly. An adhesive, such as, for example, a hot melt adhesive, an ultraviolet pressure sensitive adhesive and/or a curable adhesive may be applied to the card and/or the carrier. A layer of a second material may be applied, adhered and/or connected to the card and/or the carrier. The second material may be, for example, a laminate, such as, for example, a polypropylene or polyester film. The card may be folded onto itself to create a two-ply card. If a multi-ply card is desired, one or more ribbons may be inserted between the top side and the bottom side of the card before folding the top side of the card onto the bottom side of the card. The card may have an identifier which may be formed on or between the top side and/or the bottom side. The identifier may be, for example, a radio frequency identification tag, a smart chip, a magnetic strip or the like. The elements of the card and the mailing assembly and/or steps for producing the same may occur in a single pass manufacturing process.

It is, therefore, an advantage of the present invention to provide a system and a method for producing a card having a laminate produced from a single pass manufacturing process.

Another advantage of the present invention is to provide a system and a method for producing a laminated card removably attached to a carrier forming a mailing.

A further advantage of the present invention is to provide a system and a method for producing a card having an identification means.

A still further advantage of the present invention is to provide a system and a method for providing a durable and tear-resistant card produced from a single pass manufacturing process.

Yet another advantage of the present invention is to provide an apparatus, a system and a method for producing a laminated card having information personalized to the recipient attached to a carrier personalized for the same recipient.

Moreover, an advantage of the present invention is to provide a personalized card having a laminate attached to a personalized carrier produced from a single manufacturing pass.

And, another advantage of the present invention is to provide a multi-ply card having a laminate.

Yet another advantage of the present invention is to provide a system and a method for producing a laminated card attached to a carrier wherein the card and the carrier are personalized to a recipient.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of a card in an embodiment of the present invention.

FIG. 2 illustrates a cross-section along line I-I of the card in FIG. 1 having a laminate in an embodiment of the present invention.

FIG. 3 illustrates a plan view of a card connected to a carrier in an embodiment of the present invention.

FIG. 4 illustrates a flowchart of a method for producing a card in an embodiment of the present invention.

FIG. 5 illustrates a plan view of a system for producing a card and a carrier in an embodiment of the present invention.

FIG. 6a illustrates a top plan view of a portion of a single web of first material for producing a card and a carrier from the single web in an embodiment of the present invention.

FIG. 6b illustrates a bottom plan view of a portion of a single web of a first material for producing a card and a carrier from the single web in an embodiment of the present invention.

FIG. 7 illustrates a flowchart of a method for producing a card in an embodiment of the present invention.

FIG. 8 illustrates a flowchart of a method for producing a card in an embodiment of the present invention.

FIG. 9 illustrates a flowchart of a method for producing a card in an embodiment of the present invention.

FIG. 10 illustrates a plan view of a system for producing a card and a carrier in an embodiment of the present invention.

FIG. 11 illustrates a plan view of a system for producing a card and a carrier in an embodiment of the present invention.

### DETAILED DESCRIPTION OF THE PREFERRED EMBODIMENTS

A card and a mailing incorporating the card and a system and a method for producing the same may be formed from a single web of a first material which may be, for example, paper, cardboard and/or the like. The card may be, for example, a promotional card, an informational card, a membership card, a credit card, a discount card, a business card, a trading card, an authorization card, a coupon, a preferred customer card, an identification card, a novelty card, a game piece and/or the like. The card may be connected to, secured to and/or removably attached to a carrier to form a mailing assembly. The mailing assembly may be a direct mailing, a pamphlet, a magazine, a newspaper and/or the like.

The card may have a thickness formed from two or more layers of the first material. Each layer may be formed from the single web. An identifier may be incorporated between the layers of the card and/or on a top side and/or a bottom side of the card. The identifier may be, for example, a smart chip, a radio frequency identification tag, a magnetic strip, a bar code and/or the like. The card may have an adhesive, such as, for example, a hot melt adhesive, an ultraviolet pressure sensitive adhesive and/or a curable adhesive. The adhesive may adhere, may connect and/or may attach the card to a second material, for example a laminate, such as, for example, a polypropylene or polyester film. The card may be attached to, connected to and/or secured to a carrier which may be formed from the single web of first material.

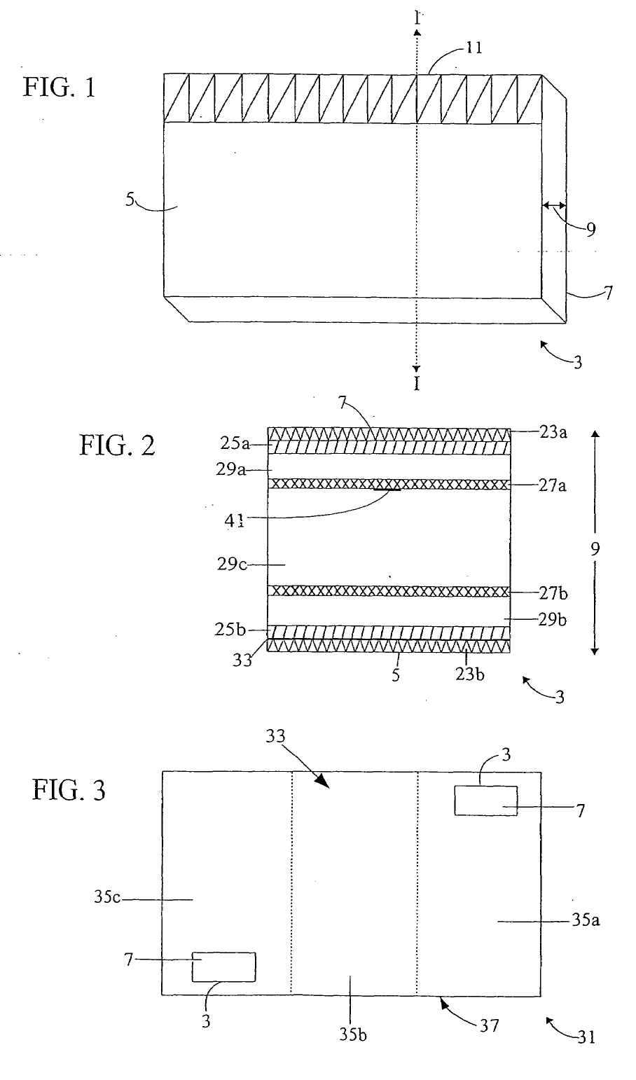

Referring now to the drawings wherein like numerals refer to like parts, FIGS. 1 and 2 illustrate a plan view and a cross-sectional view, respectively, of the card 3 in an embodiment of the present invention. The card 3 may be, for example, a promotional card, a membership card, a credit card, a discount card, a business card, a trading card, an authorization card, a coupon, a preferred customer card, an identification card, a novelty card, a ticket, a game piece and/or the like. The present invention should not be deemed as limited to a specific embodiment of the card 3. It should be understood that the card 3 may be any card known to one of ordinary skill in the art.

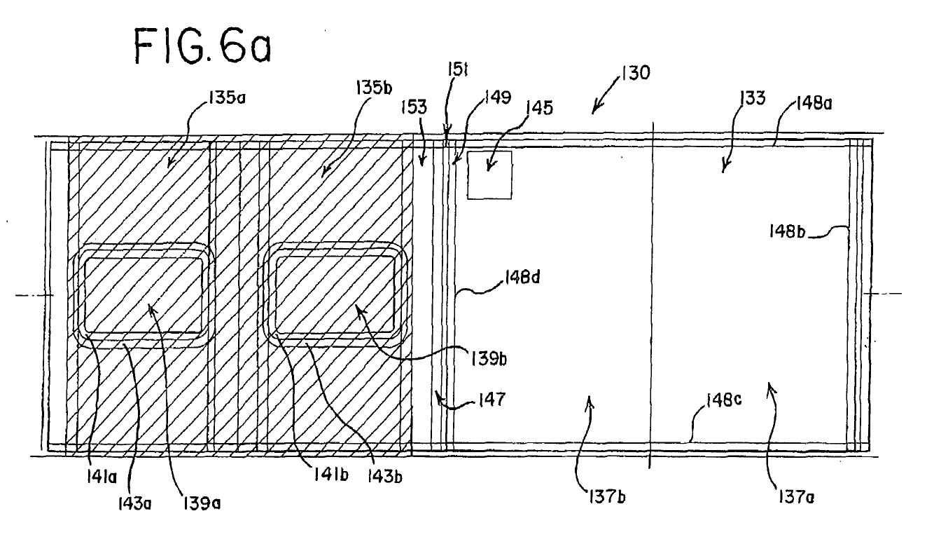

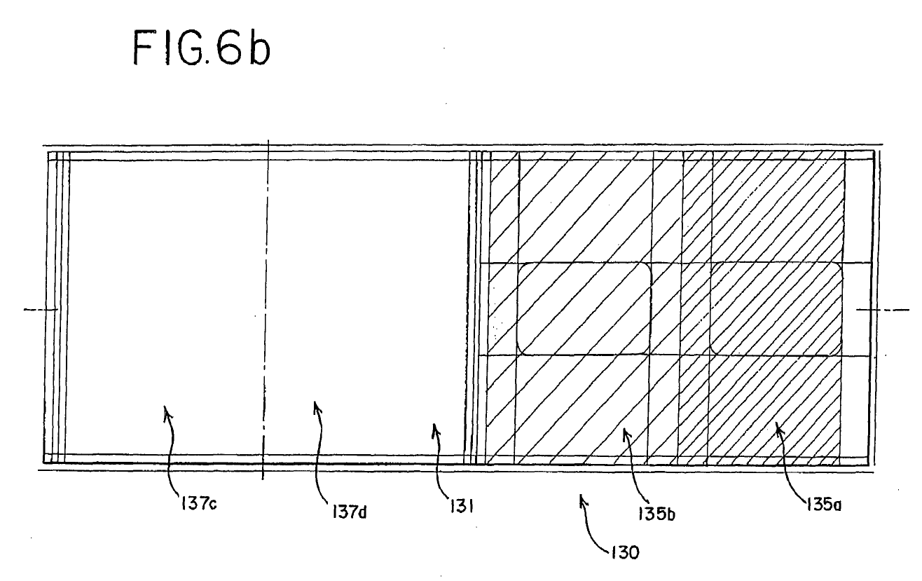

The card 3 may have a thickness 9 defined between a top side 7 and a bottom side 5 of the card 3. The card 3 may have a first layer 29a, a second layer 29b and/or a third layer 29c. The layers 29a-29c may form, for example, a three-ply card. Of course, the present invention should not be deemed limited to a specific number of layers of the card 3. The layers 29a-29c may be formed from the single web 130, as illustrated in FIG. 6a, of the first material which may be, for example, a cellulose pulp. The cellulose pulp may be, for example, paper, cardboard paper, crate paper, tissue paper and/or the like.

Indicia, information, a color, a logo and/or the like may be printed, screened and/or imaged on the layers 29a, 29b. The information may be personalized information, such as, for example, a name of a recipient, an address of the recipient, a bar code unique to the recipient and/or the like. The information may relate to a use of the card 3, such as, for example, a value of the card 3, an entity associated the card 3, a location of the card 3, a location for redeeming the card 3, an expiration date of the card 3 and/or the like.

An identifier 41 may be positioned on and/or located between the layers 29a-29c. The identifier 41 may be, for example, a smart chip, a magnetic strip, a radio frequency identification (hereinafter “RFID”) tag, a bar code and/or the like. Alternatively, the identifier 41 may be printed on the layers 29a-29c. For example, the identifier 41 may be printed with, for example, magnetic ink. The identifier 41 may store, may identify and/or may access the information. The present invention should not be limited to a specific embodiment of the identifier 41. It should be understood that the identifier 31 may be any identifier as known to one of ordinary skill in the art.

The layers 29a, 29b may connect to, may attach to, and/or may secure to the layer 29c via adhesive layers 27a, 27b, respectively, which may be formed from an adhesive, such as, for example, a glue, an epoxy and/or the like. The adhesive layers 27a, 27b may connect, may secure and/or may attach an identifier 41 to the layer 29a and/or the layer 29b.

Attached to, connected to and/or covering the layers 29a, 29b may be the adhesive layers 25a, 25b, respectively. For example, the adhesive layers 25a, 25b may be a hot melt adhesive, an ultraviolet pressure sensitive adhesive, a light curing adhesive, an ultraviolet curable adhesive and/or the like. If the adhesive layers 25a, 25b are curable adhesive layers, the adhesive layers 25a, 25b may be applied to the layers 29a, 29b in an uncured state. The adhesive layers 25a, 25b may be transparent, translucent and/or the like. The present invention should not be limited to a specific embodiment of the adhesive layers 25a, 25b. It should be understood that the adhesive layers 25a, 25b may be any adhesive layers as known to one of ordinary skill in the art. Layers 23a, 23b may attach to, may connect to, and/or may cover the adhesive layers 25a, 25b, respectively. The layers 23a, 23b may be formed from a second material, such as, for example, laminate, plastic, foil and/or the like. The laminate may be a film, such as, example, a polyester film, a polypropylene film, a polyethylene film and/or the like. The second material may prevent damage to the card 3 and/or the carrier 31 from, for example, moisture and/or may being torn.

The identifier 41 may be attached to, secured to and/or connected to the adhesive layer 25a and/or the adhesive layer 25b. The identifier 41 may attach to, may connect to and/or may cover the laminate layer 23a and/or the laminate layer 23b. Alternatively, the identifier 41 may be printed on the layers 29a-29c. The identifier 41 may be, for example, a smart chip, a magnetic strip, a radio frequency identification (hereinafter “RFID”) tag that contains a silicon chip and/or the like. The identifier 41 may store, may identify and/or may access the information.

The magnetic strip 42 may store information regarding the card 3 and/or the user of the card 3 by modifying the magnetism of iron-based magnetic particles on the magnetic strip 42. The magnetic strip may be read by physically contacting the card 3 to one of the generally known reading devices used in, for example, retail and/or sales environments.

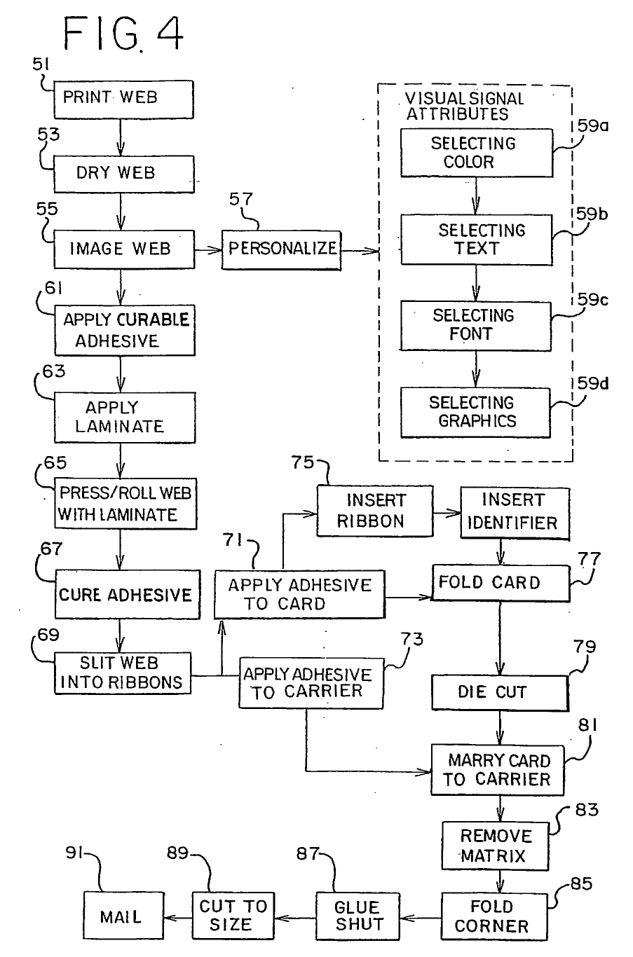

As illustrated in FIG. 3, the card 3 may attach to, may connect to, may removably attach to and/or may secure to the carrier 31 by an adhesive (not shown in the figures) to form the mailing assembly. The adhesive may be, for example, a glue, an epoxy an/or the like. In an embodiment, one or more cards may be attached to, connected to, secured to and/or removably attached to the carrier 31.

The carrier 31 may have a top surface 33 and a bottom surface 37. The top surface 33 and/or the bottom surface 37 may be printed and/or imaged with the information. Mailing information may be printed and/or imaged on, for example, the bottom surface 37 of the carrier 31. The mailing information may be an address of the recipient, an address of a sender, a promoter, an advertiser and/or the like. The information on the carrier 31 may correspond to the information displayed on the card 3.

The carrier 31 may have a first section 35a, a second section 35b and/or a third section 35c. The carrier 31 may be folded so that the first section 35a covers and/or folds onto the second section 35b. The third section 35c may cover and/or fold onto the third section 35c. As a result, the card 3 may be attached to the top surface 33 and may be enclosed, may be contained and/or may be located between sections 35a, 35b and 35c. The adhesive layers 25a, 25b may be applied to, for example, the bottom surface of the second section 35b and the third section 35c may be folded onto the second section 35b. The adhesive may secure, may connect and/or may removably attach the third section 35c to the second section 35b. As a result, the carrier 31 having a card 3 may form a mailing assembly of one or more pages. The mailing assembly may be, for example, a direct mailing, a pamphlet, a newspaper, a newsletter, a magazine and/or the like. The mailing assembly may have one or more pages and may have one or more cards removably attached to the carrier 31. The present invention should not be limited to a specific embodiment of the number of sections of the carrier 31 and/or the number of pages of the mailing assembly. It should be understood that the carrier 31 may be any number of sections and/or any number of pages as known to one of ordinary skill in the art.

As illustrated in FIG. 4, a method 41 may produce the card 3 from the single web 130 of the first material. The single web 130 may be printed by, for example, printing, imaging and/or screening a base color, such as, for example, white onto the single web 130 as shown at step 51. The single web 130 may be dried in, for example, a web press as shown at step 53. The card 3 and/or the carrier 31 may be imaged as shown at step 55. The card 3 and/or the carrier 31 may be imaged with the information, a color, a picture, an advertisement, a logo and/or the like.

The card 3 and/or the carrier 31 may be imaged with personalized information as shown at step 57. The personalized information may be, for example, the name of recipient, an address of recipient, a bar code unique to the recipient and/or preferences of the recipient as shown at steps 59a-59d, respectively. The preferences of the recipient may be, for example, a font size, a color and/or the like. The card 3 and/or the carrier 31 may be imaged simultaneously. The present invention should not be limited to a specific embodiment of the personalized information. It should be understood that the personalized information may be any personalized information as known to one of ordinary skill in the art.

The adhesive layers 25a, 25b may be formed from, for example, an ultraviolet curable adhesive which may be applied to the card 3 and/or the carrier 31 as shown at step 61. The adhesive layers 25a, 25b may be applied to the card 3 and/or carrier 31 in the uncured state. The top side 7 and/or the bottom side 5 of the card 3 may be laminated by, for example, applying a polypropylene film as shown at step 63. The polypropylene film may be applied to the uncured adhesive. The card 3 may be rolled, pressed and/or squeezed to remove air pockets which may form between the laminate layers 23a, 23b and the adhesive layers 25a, 25b as shown at step 65. The curable adhesive layers 25a, 25b may be cured by, for example, heating the adhesive, cooling the adhesive, drying the adhesive or the like as shown at step 67. Curing the adhesive layers 25a, 25b may be accomplished by, for example, ultraviolet light curing, electron beam curing and/or heat-set curing. The laminate layers 23a, 23b may be formed on the top side 7 and/or the bottom side 5 by curing the adhesive layers 25a, 25b, respectively.

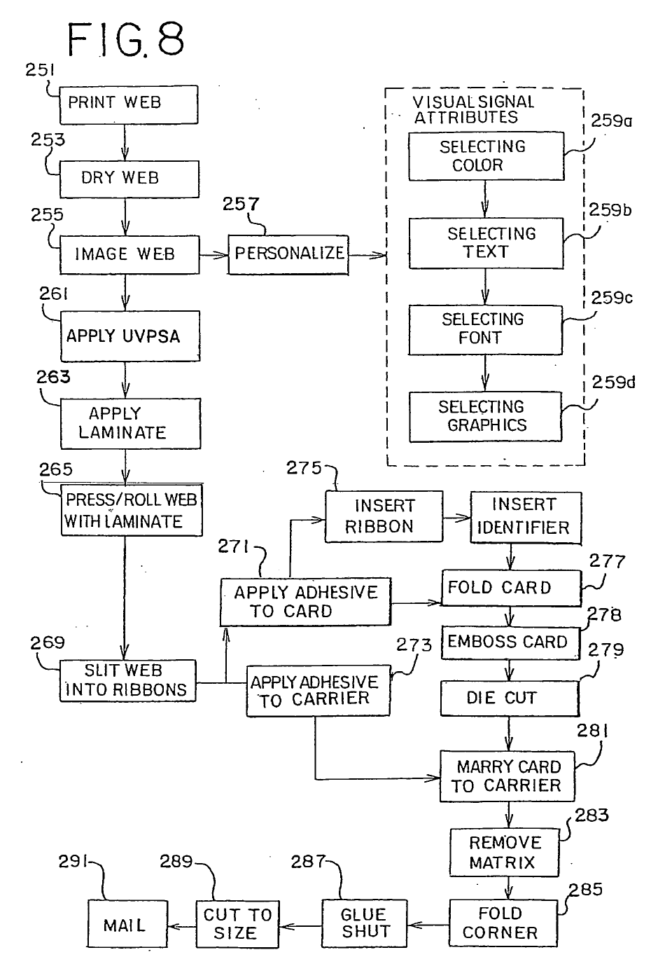

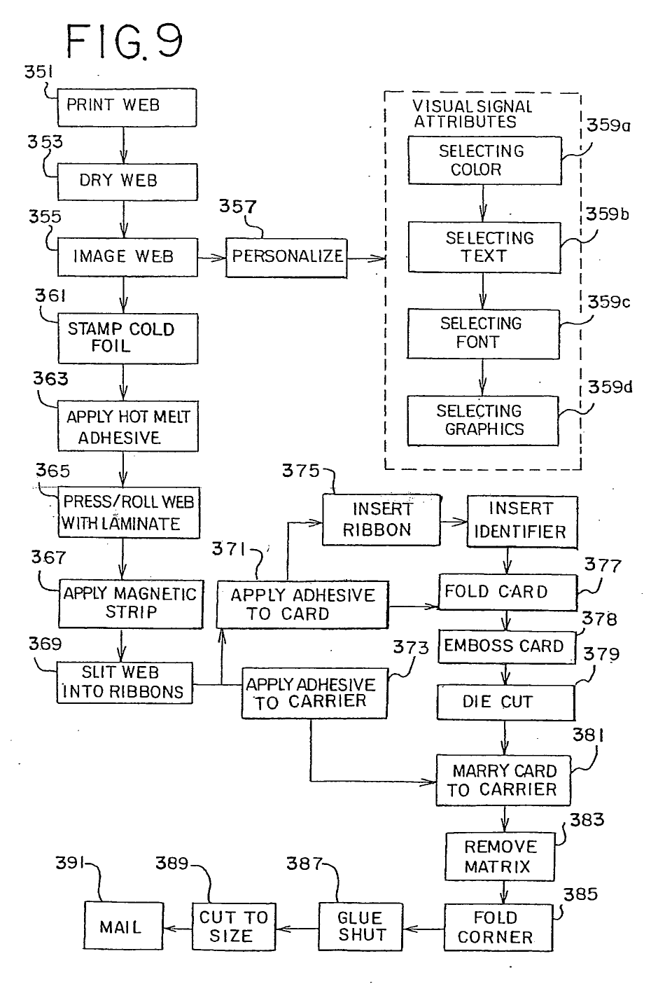

Alternatively, the adhesive layers 25a, 25b may be formed from, for example, an ultraviolet pressure sensitive adhesive (“UVPSA”) which may be applied to the card 3 and/or the carrier 31 as shown in FIG. 8 at step 261. The adhesive layers 25a, 25b of UVPSA may be applied to the card 3 and/or carrier 31 using, for example, a flexo applicator, or may be extruded on the card 3 and/or the carrier 31 using an ultraviolet curable extruding process.

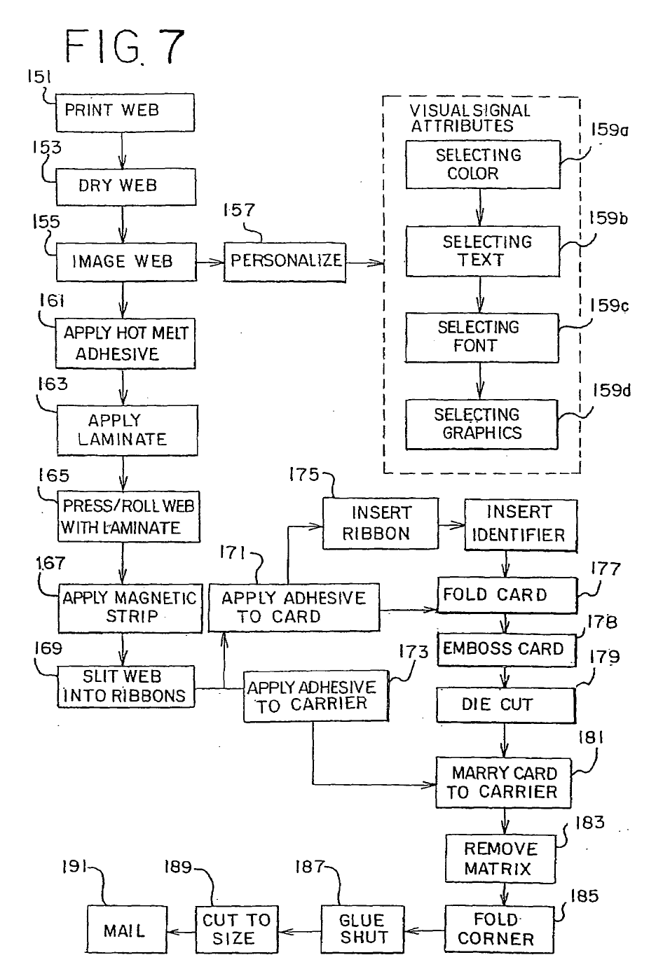

Alternatively, the adhesive layers 25a, 25b may be formed from a hot melt adhesive which may be applied to the card 3 and/or the carrier 31 as shown in FIG. 7 at step 161. The adhesive layers 25a, 25b may be applied to the card 3 and/or the carrier 31 using, for example, a slot applicator or a roll coater.

Embodiments using a UVPSA and/or a hot melt adhesive may have the top side 7 and/or the bottom side 5 of the card 3 laminated by, for example, applying a polypropylene or a polyester film. The film may be applied to the adhesive. The card 3 may be rolled, pressed and/or squeezed to remove air pockets which may form between the laminate layers 23a, 23b and the adhesive layers 25a, 25b.

The single web 130 may have one or more cards and/or one or more carriers printed and/or imaged on the single web 130. The single web 130 may be cut, slit and/or torn into one or more ribbons as shown at step 69. One or more of the ribbons may be used to form the card 3 and/or the carrier 31.

The adhesive layers 27a, 27b may be applied to the bottom side 5 and/or the top side 7 of the card 3 as shown at step 71. For example, the laminated layers 23a, 23b may be applied to one side of the single web 130 and the adhesive layers 27a, 27b may be applied to the opposite side of the single web 130. The single web 130 may be folded onto itself to contain and/or to enclose the adhesive layers 27a, 27b between the top side 7 and the bottom side 5 of the card 3.

One or more ribbons may be inserted between the top surface 7 and/or the bottom surface 5 of the card 3 prior to folding the card 3 as shown at step 75. Each ribbon may be secured to, attached to and/or connected to the top side 7 and/or the bottom side 5 via the adhesive layers 27a, 27b which may form, for example, the multi-ply card. Further, the identifier 41 may be inserted on and/or positioned between the top side 7 and/or the bottom side 5 of the card 3, as shown at step 76. The present invention should not be limited to a specific embodiment of the location of the identifier 41. The identifier 41 may be secured to, connected to and/or removably attached to any layer of the card 3 that may be apparent to one of ordinary skill in the art.

As illustrated in FIG. 4, the magnetic strip 42 may be applied to the card 3. As generally shown at step 66, the card 3 may be processed by a magnetic strip unit wherein film that includes the magnetic strip 42 may be applied to the card 3. Alternatively, the magnetic strip 42 may be heat sealable to allow application of the magnetic strip 42 to the card 3. In another embodiment, the laminate may contain the magnetic strip 42 and allow the magnetic strip 42 to be applied with the laminate layers 23a, 23b.

The single web 130 may be folded by, for example, folding the top side 7 onto and/or over the bottom side 5 of the card 3 as shown at step 77, as illustrated in FIG. 4. The card 3 may be cut by, for example, die cutting from the single web 130 as shown at step 79. The card 3 may remain attached to, connected to and/or within the matrix.

The adhesive layers 27a, 27b may be applied to the bottom surface 37 and/or the top surface 33 of the carrier 31 as shown at step 73. The card 3 may be positioned on, secured to and/or removably attached to the adhesive of the carrier 31 as shown at step 81. One or more cards may be attached to, connected to and/or secured to the carrier 31. The matrix may be removed from the card 3 by pulling, tearing and/or suctioning as shown at step 83. For example, a vacuum system may suction the card 3 from the carrier 31 and/or the matrix.

The card 3 may be folded as generally shown at step 77 and then may be embossed, as shown in FIG. 7 at step 178. The card 3 may be embossed by an embossing unit 318, such as, for example, a male/female die wherein the web 130 is processed by the embossing unit 318. Personal information may be embossed on the card 3, such as, for example, the name of recipient, an address of recipient, a bar code unique to the recipient and/or the like. The bar code may present a visual format that may be read by known machines to scan the bar code. A number may be embossed on the card wherein the number, for example, may relate to a value of the card 3, an entity associated the card 3, a location of the card 3, a location for redeeming the card 3, an expiration date of the card 3 and/or the like.

A number may be embossed on the card 3 wherein the same number is embossed on more than one card 3. Alternatively, the number embossed on the card 3 may be variable. A variable number may be embossed on each one of the cards 3 by using a numbering head. Such numbering heads are made by Atlantic Zeiser or Paul Leibinger Numbering Machine L.P.

The carrier 31 may be folded as shown in FIG. 4 at step 85. The carrier 31 may be folded one or more times. The adhesive layers 27a, 27b may be applied to the carrier 31 to secure and/or to removably attach the top surface 33 to the bottom surface 37 of the carrier 31 as shown at step 87. The carrier 31 may be cut to a size by, for example, a rotary cutter as shown at step 89. The size may be the size desired by the recipient, the advertiser and/or the promoter. The carrier 31 having the card 3 may be prepared for mailing by, for example, weighing the card 3 and the carrier 31 and/or applying postage to the carrier 31, as shown at step 91.

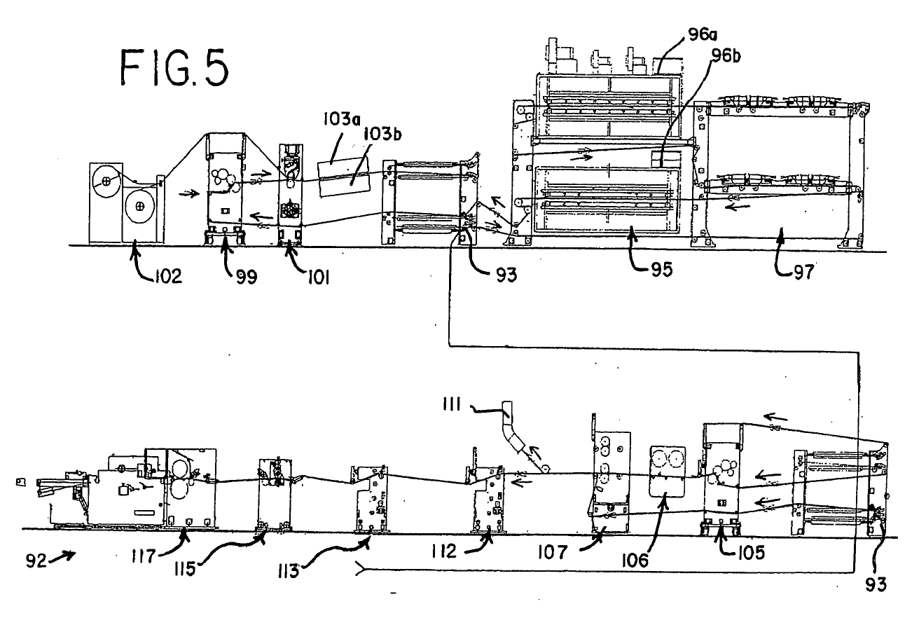

A system 92 may produce the card 3 and the carrier 31 in a single-pass manufacturing process in an embodiment of the present invention as shown in FIG. 5. The single web 130 may be printed and/or dried before passing over a bar 93 which may guide, position and/or move the single web 130 to a transporter 95. The bar 93 may be, for example, an idler, an anglebar and/or the like. The bar 93 may align, may steer and/or may position the single web 130 with respect to the transporter 95 and/or a imager 97.

The transporter 95 may move, may slide, may push and/or may pull the single web 130 from the bar 93 to the imager 97. The transporter 95 may position the single web 130 by, for example, flipping, turning and/or rotating the single web 130 before the single web 130 may enter the imager 97. The transporter 95 may have, for example, idlers, conveyor belts, rollers and/or the like. Dryers 96a, 96b may be attached to, secured to and/or connected to the transporter 95. The dryers 96a, 96b may be, for example, infrared dryers and/or the like for heating the single web 130 and/or removing moisture from the single web 130.

The single web 130 may be imaged and/or printed on the single web 130. The single web 130 may return to the image transporter 95 to be dried by the dryers 96a, 96b. The image transporter 95 may move the single web 130 through a laminator 101 to an adhesive applier 99 which may, for example, apply the adhesive layers 25a, 25b. The single web 130 may return to the laminator 101 to apply the second material to the single web 130. The second material may be provided from an unwinder 102 which may, for example, continuously supply the second material to the laminator 101.

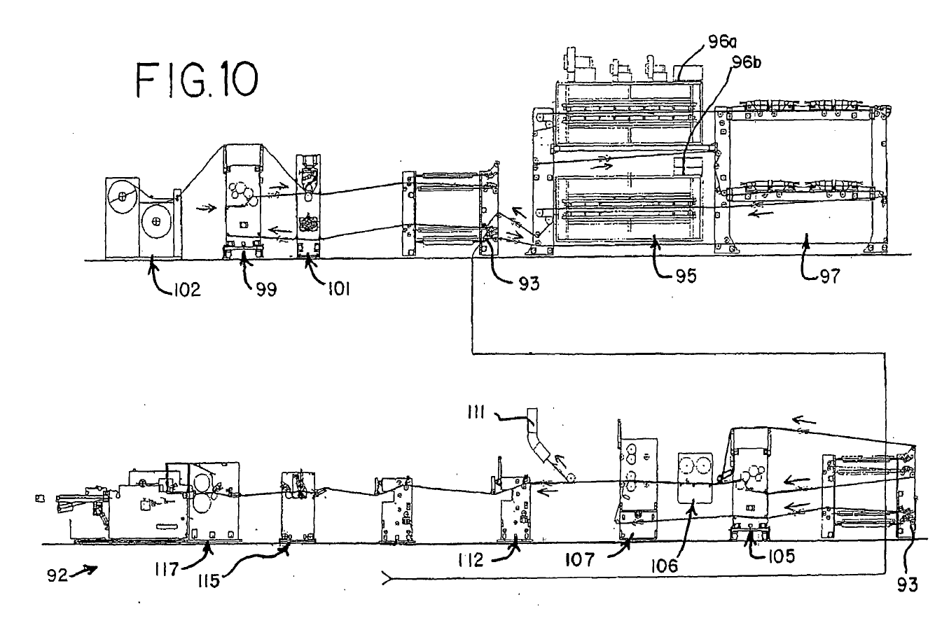

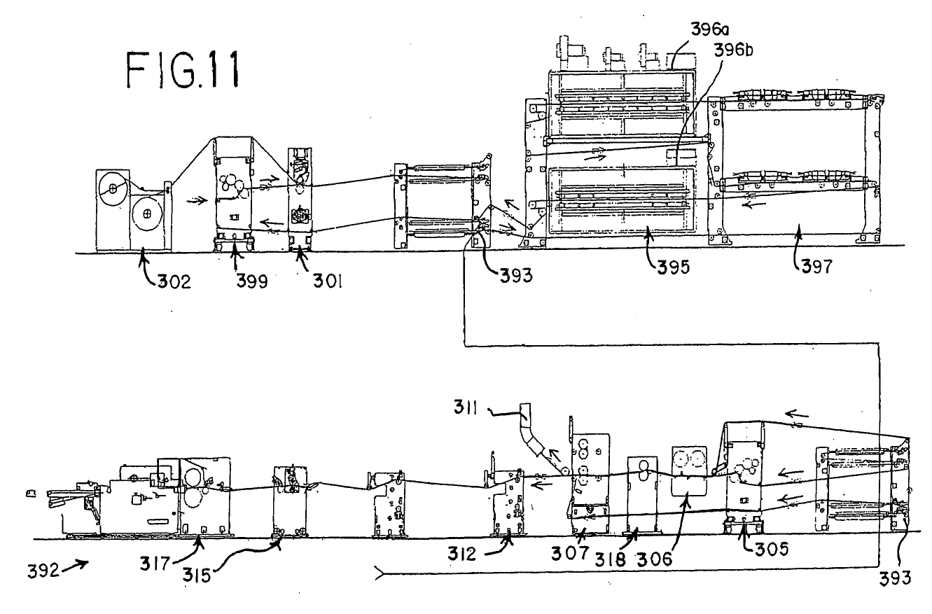

In an embodiment using the curable adhesive, the single web 130 may move, may pass and/or may be directed from the laminator 101 to curing lamps 103a, 103b, such as, for example, ultraviolet curing lamps. The curing lamps 103a, 103b may cure and/or may dry the curable adhesive layers 25a, 25b through the second material. The present invention should not be limited to a specific embodiment of the curing lamps 103a, 103b. It should be understood that the curing lamps 103a, 103b may be any curing lamps as known to one of ordinary skill in the art. In an embodiment using an adhesive that may not be cured, curing lamps 103a, 103b may not be needed in the system, as shown in FIG. 10.

The single web 130 may be cut and/or separated into two or more ribbons (not shown in the drawings) as the single web 130 passes from the bar 93 to a first gluer 105. In an embodiment the single web 130 may be slit, may be separated and/or may be cut into a first ribbon, a second ribbon and a third ribbon. The first ribbon may be used to form the carrier 31. The second ribbon may be used to form the card 3. The third ribbon may be inserted between the top side 7 and the bottom side 5 of the card 3. Each of the ribbons may pass and/or move through the first gluer 105 which may apply an adhesive to each ribbon. For example, the first gluer 105 may apply the adhesive to form the adhesive layers 27a, 27b of the card 3.

The third ribbon may be the layer 29c, as illustrated in FIG. 2. The adhesive layers 27a, 27b may adhere and/or attach the third ribbon to the first ribbon and/or the second ribbon to form a three-layer card. The second ribbon, for example, may be folded onto itself one or more times to create additional layers of the card 3.

The second ribbon, for example, may pass through an applicator 106 which may apply and/or insert the identifier 41. The first gluer 105 may apply the adhesive layers 27a, 27b to the second ribbon to adhere and/or to secure the identifier 41 to the card 3. The identifier 41 may be inserted before or after the second ribbon may be folded onto itself and/or before the third ribbon may be inserted between the top side 7 and the bottom side 5 of the card 3.

The second ribbon may be cut by, for example, die cutting the card 3 from, for example, the second ribbon at the die cutter 107. The card 3 may remain attached to, connected to and/or removably secured to the matrix. The second ribbon and the first ribbon may be matched, married, secured, removably attached and/or connected at or after the die cutter 107. The second ribbon and the first ribbon may form one or more mailing assemblies which may move over, under and/or through an idler 109. The matrix may be removed from one or more mailing assemblies to form the card 3 and the carrier 31. The matrix may be removed from the single web 130 via a suction 111 which may be, for example, a vacuum suction.

The card may be embossed by an embossing unit 318 as generally illustrated in FIG. 11. The embossing unit 318 may have, for example, a male/female die wherein the web 130 may pass through the embossing unit 318 and may be embossed using the male/female die. Personal information and/or a number may be embossed on the card 3. A number may be embossed on the card 3 wherein the same number may be embossed on more than one card 3. Alternatively, the number embossed on the card 3 may be variable. A variable number may be embossed on each card 3 by using a numbering head.

An additional ribbon 150 may be provided on the card 3 and/or carrier 31. The additional ribbon 150 may be added to the card 3 and/or the carrier 31 before the laminate # is applied. The adhesive # may be applied to the single web of material 130 before and/or after the additional ribbon 150 may be added to the card 3 and/or the carrier 131. The additional ribbon 150 may form a middle layer of a three-ply card by a method that may have the roll stand 102 feeding the additional ribbon 150 onto the single web of material 130, and a folder 103 may fold the single web of material 130 onto and/or over the additional ribbon 150. Therefore, the card 3 may be formed using the additional ribbon 150 as the third layer 29c and forming the first layer 29a and the second layer 29b from the single web of material 130 folded onto and/or over the additional ribbon 150 as generally illustrated in FIG. 2.

Furthermore, using an additional ribbon 150 that is a cold foil 293, the card 3 may undergo inline cold foil stamping as illustrated in FIG. 9. As generally shown at step 361, the cold foil 293 may be applied onto the card 3 and/or the carrier 31. The cold foil 293 may have a patterned layer 294 and a substrate layer 295. The patterned layer 294 may have a visual image of colors, images, numbers, text and/or the like. The cold foil 293 may be applied onto the card 3 and/or the carrier 31. The substrate layer 295 of the cold foil 293 may be pulled from the card 3 and/or the carrier 31 to transfer the patterned layer 294 from the cold foil 293 to the card 3 and/or the carrier 31. After cold foil stamping, the adhesive, such as, for example, hot melt adhesive, may be applied at step 363, and the laminate may be applied at step 365.

A folder 112 may fold the sections 35a-35c, as illustrated in FIG. 3, of the carrier 31 to form the mailing assembly. For example, each of the carriers 31 may be folded one or more times to form a multi-page mailing assembly. The mailing assembly may pass through a second gluer 113 which may close, may seal and/or may shut the multi-page mailing. A slitter 115 may separate one or more mailing assemblies into individual mailing assemblies. Each mailing assembly may then be cut to the size by, for example, a trimmer 117 which may trim, may cut and/or may edge the carrier 31.

As illustrated in FIGS. 6a and 6b, the top portion 131 of the single web 130 may be opposite to the bottom portion 133 of the single web 130. The top portion 131 and the bottom portion 133 of the web 130 may form the card 3 and/or the carrier 31. The top side 7 and the bottom side 5 of the card 3 may be imaged and/or printed by the imager 97. Areas 135a, 135b of the single web 130 may form the card 3. Areas 139a, 139b of the single web 130 may define the top side 7 and the bottom side 5 of the card 3 and may be imaged and/or printed by the imager 97, as illustrated in FIG. 5. The areas surrounding the top side 7, the bottom side 5 and areas 139a, 139b may not receive imaging. The areas 139a, 139b or a portion of the areas 139a, 139b may be cut away from the single web 130 at the die-cutter 107, as illustrated in FIG. 5. The die-cutter 107 may cut within areas 141a, 141b without cutting imaging and/or information on the card 3. For example, the areas 141a, 141b may be cut and/or removed by the die-cutter 107 to form the card 3 which may be imaged and/or printed on the top side 7 and/or the bottom side 5. Areas 143a, 143b of the single web 130 absorb and/or receive ink, dye and/or the like that may leak and/or flow from the areas 139a, 139b.

The adhesive layers 25a, 25b and/or the laminate layers 23a, 23b may be applied to the bottom portion 133 and/or the top portion 131 of the web 130, as illustrated in FIGS. 6a and 6b. The adhesive layers 27a, 27b may be applied on the areas 135a, 135b of the web 130. In an embodiment, the adhesive layer 27a may be applied to the area 135a and/or the adhesive layers 25a may be applied on the area 135b of the web 130.

Areas 137a, 137b of the single web 130 may form, for example, a first page and/or a fourth page of the carrier 31. The areas 137c, 137d may form, for example, a second page and/or a third page of the carrier 31. The areas 137a-137d may be imaged, screened and/or printed at the steps 55, 57 and/or 59a-59d, as illustrated by FIG. 4. Area 145 of the single web 130 may be imaged, printed, stamped with, for example, the postage required for sending and/or mailing the mailing assembly. Area 147 of the single web 130 may be cut, trimmed and/or removed to form edges 148a-148d of the carrier 31.

The area 149 of the single web 130 may be imaged, may be screened and/or may be printed so that the carrier 31 may have imaging and/or printing to the edges 148a-148d of the carrier 31. Area 151 may receive ink, dye and/or the like which may leak, bleed and/or flow from the imager 97. The area 151 may prevent imaging and/or printing on the carrier 31 from being cut and/or removed from the carrier 31.

The area 135a may be folded over and/or onto the area 135b to form the top side 7 and the bottom side 5 of the card 3 within the matrix. An area 153 may be cut, trimmed and/or removed to separate the areas 135a, 135b from the areas 137a, 137b.

The card 3 may be adhered, secured and/or removably attached to the carrier 31 at, for example, the area 137d. For example, the area 137a may be folded over and/or onto the area 137d to form a mailing assembly having four pages. The areas 137a-137d may form the first pages, the second page, the third page and the fourth page, respectively.

The mailing assembly is formed by removably attaching, connecting and/or securing the card 3 to the carrier 31. The card 3 and/or the carrier 31 may be produced from the single web 130 of material which may be, for example, paper, cardboard, cray paper or the like. One or more cards and/or one or more carriers may be formed from the single web 130 of material. The card 3 may be, for example, a promotional card, the membership card, the game piece, the credit card and/or the like. The carrier 31 may be folded to create more than one page. The card 3 may be attached to the carrier 31 via the adhesive layers 27a, 27b. The card 3 and/or the carrier 31 may be printed, imaged and/or screened. The card 3 and/or the carrier 31 may be personalized to the recipient of the mailing. The adhesive layers 25a, 25b may be applied to the top side 7 and/or the bottom side 5 of the card and/or the carrier 31. The card 3 and/or the carrier 31 may be laminated by, for example, applying the polypropylene or polyester film. The card 3 may be folded onto itself to create the two-ply card. If the multi-ply card is desired, the ribbon or the ribbons may be inserted in between the top side 7 and the bottom side 5 of the card 3 before folding the top side 7 and the bottom side 5 of the card 3 onto each other. The card 3 may have the identifier 41 which may be formed on and/or located between the top side 7 and/or the bottom side 5. The identifier 41 may be, for example, the radio frequency identification tag, the smart chip, the magnetic strip and/or the like.

It should be understood that various changes and modifications to the presently preferred embodiments described herein will be apparent to those skilled in the art. Such changes and modifications may be made without departing from the spirit and scope of the present invention and without diminishing its attendant advantages.

I claim:

1. A method for manufacturing a mailing assembly, the method comprising the steps of:
printing at least one card and at least one carrier on a single web of material wherein the card has a top surface and a bottom surface wherein the card has a top layer and bottom layer;
applying a first adhesive to the single web of material;
applying a laminate to the first adhesive;
applying a second adhesive to the carrier, wherein the second adhesive attaches the laminate of the card to the carrier;
removing the card and the carrier attached to the card from the ribbon; and
folding the carrier over the card wherein the card resides between the top surface of the carrier and the bottom surface of the carrier.

2. The method of claim 1 further comprising the step of:
cutting the single web of material into a plurality of ribbons wherein a first ribbon forms a top layer of the card, a second ribbon forms the bottom layer of the card and a third ribbon forms the carrier.

3. The method of claim 1 further comprising the step of:
folding the card wherein folding the card forms the top layer of the card and the bottom layer of the card.

4. The method of claim 1 wherein at least two cards are printed on the single web of material and the cards are imaged with personalized information so that a first card is different from a second card.

5. The method of claim 1 further comprising the step of:
attaching an identifier to the card wherein the identifier is information related to a user of the card.

6. The method of claim 1 further comprising the step of:
attaching an identifier to the card wherein the identifier is information related to a value of the card.

7. The method of claim 1 wherein the first adhesive is a hot melt adhesive.

8. The method of claim 1 wherein the first adhesive is an ultraviolet pressure sensitive adhesive.

9. The method of claim 1 wherein the first adhesive is a curable adhesive.

10. The method of claim 1 further comprising the step of:
pressing the single web of material to remove air pockets in the laminate.

11. The method of claim 1 further comprising the step of:
embossing the card wherein the single web of material is processed by an embossing unit having a die to emboss the card.

12. The method of claim 1 further comprising the step of:
cold foil stamping the card to transfer a layer from a sheet of foil onto the card.

13. A method for manufacturing a mailing assembly, the method comprising the steps of:
printing at least one card and at least one carrier on a first sheet constructed of a first material wherein the carrier has a top surface and a bottom surface wherein the card has a top layer and bottom layer;
applying a first adhesive to the first sheet;
applying laminate to the first adhesive;
applying a second sheet on the card wherein the card is folded over the second sheet to form the top layer of the card and the bottom wherein the second sheet is constructed of a second material;
applying a second adhesive to the carrier wherein the second adhesive attaches the laminate of the card to the carrier; and
removing the card and the carrier attached to the card from the ribbon.

14. The method of claim 13 further comprising the step of:
folding the carrier over the card wherein the card resides between the top surface of the carrier and the bottom surface of the carrier.

15. The method of claim 13 wherein the second material is the same material as the first material.

16. The method of claim 13 further comprising the step of:
selecting visual attributes for the card wherein the visual attributes are printed on the card.

17. The method of claim 13 further comprising the step of:
embossing a number on the card.

18. The method of claim 13 further comprising the step of:
printing at least two cards and embossing a plurality of numbers on the cards wherein the number on each of the cards is a different number.

19. A mailing assembly comprising:
a card wherein the card has a top card layer and a bottom card layer further wherein the top card layer and the bottom card layer are made from a same material;
an identifier between the top card layer and the bottom card layer;
a first adhesive layer between the top card layer and the bottom card layer;
a top laminate layer attached to the top card layer;
a bottom laminate layer attached to the bottom card layer; and
a carrier removably attached to the card wherein a first carrier section is folded over the card so that the card resides between the first carrier section and a second carrier section.

20. The mailing assembly of claim 19 wherein the identifier is a radio frequency identification tag that contains a silicon chip that stores information.

21. The mailing assembly of claim 19 wherein the identifier is a bar code that provides a machine-readable visual format.

22. The mailing assembly of claim 19 further comprising:
an identifier attached to the card wherein the identifier is information related to a user of the card.

23. The mailing assembly of claim 19 further comprising:
an identifier attached to the card wherein the identifier is information related to a value of the card.

24. The mailing assembly of claim 19 further comprising:
an identifier attached to the card wherein the identifier is information related to a location where the card is redeemed.

25. The mailing assembly of claim 19 further comprising:
an identifier attached to the card wherein the identifier is information related to a location of the card.

26. The mailing assembly of claim 19 wherein the top laminate layer is a polyester film.

27. The mailing assembly of claim 19 wherein the top laminate layer is a polypropylene film.

28. The mailing assembly of claim 19 further comprising:
a second adhesive layer between the top card layer and the top laminate layer.

29. A mailing assembly comprising:
a card having a top card layer, a middle card layer and a bottom card layer further wherein the top card layer, the middle card layer and the bottom card layer are made from a same material;
a first adhesive layer between the top card layer and the middle card layer;
a second adhesive layer between the middle card layer and the bottom card layer;
a top laminate layer attached to the top card layer;
a third adhesive layer between the top laminate layer and the top card layer;
a bottom laminate layer attached to the bottom card layer; and
a fourth adhesive layer between the bottom laminate layer and the bottom card layer.

30. The mailing assembly of claim 29 further comprising:
a number embossed on the card.

31. A system for manufacturing a mailing assembly, the system comprising:
a transporter that positions and moves a single web of material to an imager that prints a plurality of cards and a plurality of carriers on a single web of material wherein each one of the plurality of cards has a top card layer and a bottom card layer;
a laminator that receives the single web of material from the imager and applies a laminate to the single web of material;
a first gluing unit that receives the single web of material from the laminator and uses an adhesive to attach the top card layer to the bottom card layer;
a second gluing unit that receives the single web of material from the first gluing unit and attaches one of the plurality of carriers to at least one of the plurality of cards.

32. The system of claim 31 further comprising:
a dryer that receives the single web of material from the imager and removes moisture from the single web of material.

33. The system of claim 31 further comprising:
a roller stand that feeds a second web of material onto the single web of material so that the single web of material is folded over the second web of material.

34. The system of claim 31 further comprising:
a die cutter that cuts the single web of material into at least two ribbons so that a first ribbon forms the top card layer, a second ribbon forms the bottom card layer and a third ribbon forms one of the plurality of carriers wherein the die cutter passes the ribbons to the first gluing unit.

35. The system of claim 31 further comprising:
a die cutter that cuts the single web of material into at least two ribbons so that a first ribbon forms the top card layer, a second ribbon forms the bottom card layer and a third ribbon forms a middle card layer and the die cutter passes the ribbons to the first gluing unit.

36. The system of claim 31 further comprising:
an applicator that receives the single web of material from the laminator and inserts an identifier between the top card layer and the bottom card layer wherein the identifier provides information related to a user of the card.

37. The system of claim 31 further comprising:
an applicator that receives the single web of material from the laminator and inserts an identifier between the top card layer and the bottom card layer wherein the identifier provides information related to a value of the card.

38. The system of claim 31 further comprising:
an applicator that inserts an identifier between the top card layer and the bottom card layer and passes the single web of material to the laminator.

39. The system of claim 31 further comprising:
a folder that receives the single web of material from the imager and folds a portion of the single web of material to form the top card layer and the bottom card layer.

40. The system of claim 31 further comprising:
a folder that receives the single web of material from the laminator and folds a portion of the single web of material to form the top card layer and the bottom card layer.

41. The system of claim 31 further comprising:
a curing unit that receives the single web of material from the first gluing unit and cures the adhesive.

42. The system of claim 31 further comprising:
a trimmer that receives the single web of material from the second gluing unit and trims the plurality of carriers.

43. The system of claim 31 further comprising:
an embossing unit that has a die to emboss the card.

44. The system of claim 31 further comprising:
a cold foil stamping unit that transfers a layer from a sheet of foil onto the card.

45. The system of claim 31 further comprising:
a suction unit that removes a portion of the single web of material that does not form one of the plurality of cards or one of the plurality of carriers.
